(19) United States
(12) Patent Application Publication
(10) Pub. No.: US 2010/0190968 A1
(43) Pub. Date: Jul. 29, 2010

(54) OLEANANE TRITERPENE SAPONIN COMPOUNDS WHICH ARE EFFECTIVE ON TREATMENT OF DEMENTIA AND MILD COGNITIVE IMPAIRMENT (MCI) AND IMPROVEMENT OF COGNITIVE FUNCTION

(76) Inventors: Bong Cheol Kim, Gyeonggi-do (KR); Wonrack Choi, Seoul (KR); Soo Min Lee, Seoul (KR); Se Jun Yun, Seoul (KR); Chang-Kyun Han, Seoul (KR); Guang-Jin Im, Gyeonggi-do (KR); Wie-Jong Kwak, Seoul (KR)

Correspondence Address: FROMMER LAWRENCE & HAUG, 745 FIFTH AVENUE- 10TH FL., NEW YORK, NY, 10151, US

(21) Appl. No.: 12/446,523

(22) PCT Filed: Oct. 24, 2006
(86) PCT No.: PCT/KR06/04355

§ 371 Date: Apr. 21, 2009

(51) Int. Cl. C07H 15/256

(52) U.S. Cl. 536/18.1

(57) ABSTRACT

The present invention relates to the use of oleanane-type triterpene saponin compounds, which are effective for improving memory and learning ability, as an effective ingredient of drugs for the treatment and prevention of dementia and mild cognitive impairment and health foods for the improvement of brain functions including cognitive function.

### TECHNICAL FIELD

The present invention relates to a use of an oleanane-type triterpene saponin compound represented by the formula (1) below, which is effective for improving memory and learning abilities, as an effective ingredient of health food for the treatment and prevention of dementia and mild cognitive impairment and the improvement of brain functions including a cognitive function:

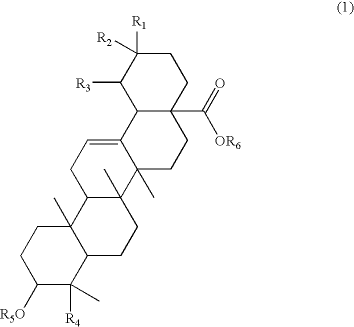

wherein each of R₁, R₂ and R₃ is hydrogen or C₁-C₄ alkyl; R₄ is C₁-C₄ alkyl or C₁-C₄ hydroxyalkyl; and each of R₅ and R₆ is hydrogen or sugar, wherein at least one of R₅ and R₆ is sugar which is selected from glucose, galactose, rhamnose, xylose, arabinose and glucuronic acid.

### BACKGROUND ART

Triterpene saponin refers to a sapogenin (aglycone) compound having a triterpene group and forming a glycoside bond or ester bond with a sugar or sugar chains. Triterpene saponins are classified based on the type of sapogenins. Typical examples of triterpene saponins are saponins having pentacyclic triterpene groups, for example, oleanane, ursane, lupane, hopane, taraxerane, and the like. Also, saponins are classified depending on their binding sites with sugar chains. To take oleanolic acid for example, one bonded with a sugar chain at either the C-3 or C-28 position is called monodesmoside while one bonded with sugar chains at both C-3 and C-28 positions is called bisdesmoside. Glycoside bonding predominates at the C-3 position and ester bonding predominates at the C-28 position. The sugars that typically bind with saponin are glucose, galactose, rhamnose, xylose, arabinose, glucuronic acid, and the like.

The absorption rate of saponins by intestines is generally low when they are present in the form of bisdesmoside. However, the ester bond present at C-28 position of saponins is easily hydrolyzed by the enzymes of intestinal bacteria. As the sugar chain is removed, the sugar chain terminal connected to the C-3 position by glucoside bond is partially hydrolyzed beginning from the terminal and then absorbed by the body [Kim D H, Bae E A, Han M J, Park H J, Choi J W. Metabolism of Kalopanaxsaponin K by human intestinal bacteria and antirheumatoid arthritis activity of their metabolites. Biol Pharm Bull. 2002 January; 25(1): 68-71.]. In contrast, the triterpene sapogenin itself, not being bound to sugar, has low solubility and shows reduced activity in animal tests because of its relatively low intestinal absorption, compared with that of the glucoside counterpart [Yoshikawa M, Matsuda H. Antidiabetogenic activity of oleanolic acid glycosides from medicinal foodstuffs. BioFactors. 2000; 13(1-4): 231-7].

As the population of aged people increases throughout the world various kinds of degenerative geriatric diseases have been raised as social as well as economic issues. According to the statistics released by the Alzheimer's Association and the National Institute on Aging, about 4 million Americans are suffering from dementia. Although it is common that dementia develops at the age of 60 or later, in rare cases, it begins even in the 50s. Of all Americans of 65 years or older, 10.3% are suffering from dementia and as much as 95 billion dollars is spent each year to treat dementia.

According to a report from the Korea Institute for Health and Social Affairs, the population of dementia patients has been increasing rapidly along with the increase in the number of aged people. The population of dementia patients aged 65 or older is expected to increase to 9% in 2020, 0.7% up from 8.3% in 1995. And, according to the estimate by the Korea National Statistical Office, the population of the aged people suffering from dementia is expected to increase from 277,048 (8.3% of the people aged 65 years or older) in 2000 to 527,068 (9%) in 2015 and 619,132 (9%) in 2020. Since dementia is an intractable illness that devastates the patient's life and destroys the lives of the patient's family, it is becoming a serious social, economical problem and has to be overcome.

Dementia may develop due to a variety of causes, and its main symptom is characterized by having markedly reduced learning ability and memory.

Tacrine, the first treatment for dementia approved by the FDA in 1993, can postpone the loss of cognitive function in about 30% of patients with Alzheimer's disease at its early or middle stage by inhibiting the breakdown of acetylcholine (ACh) in the brain. However, it is hardly used these days because it generates a lot of adverse reactions against the liver. Aricept, which was approved by the FDA in 1996, is used to increase the level of acetylcholine. This drug can be taken once a day before sleeping. Its common side effects include nausea, diarrhea and tiredness, which do not last long. Accordingly, development of a new treatment for dementia with good efficacy and few side effects is required.

The present inventors have performed intensive researches to develop a substance effective for improving memory and learning abilities with little toxicity. As a result, they discovered that an oleanane-type triterpene saponin compound is effective for treating dementia and mild cognitive impairment and improving cognitive function.

Accordingly, an object of the present invention is to provide a therapeutic pharmaceutical drug and a health food for the improvement of brain functions comprising an oleanane-type triterpene saponin compound as an effective ingredient.

### [Disclosure]

The present invention relates to a therapeutic pharmaceutical drug for treating dementia and mild cognitive impairment or a health food for the improvement of brain functions comprising the oleanane-type triterpene saponin compound represented by the following formula (1):

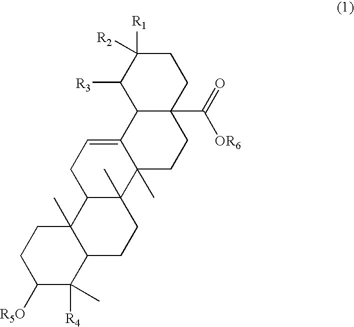

wherein each of R₁, R₂ and R₃ is hydrogen or C₁-C₄ alkyl; R₄ is C₁-C₄ alkyl or C₁-C₄ hydroxyalkyl; and each of R₅ and R₆ is hydrogen or sugar, wherein at least one of R₅ and R₆ is sugar which is selected from glucose, galactose, rhamnose, xylose, arabinose and glucuronic acid.

Hereunder is given a more detailed description of the present invention.

Of the oleanane-type triterpene saponin compounds represented by the formula (1), oleanolic acid saponin represented by the formula (1a) below and hederagenin saponin represented by the formula (1b) below are well known in the related art:

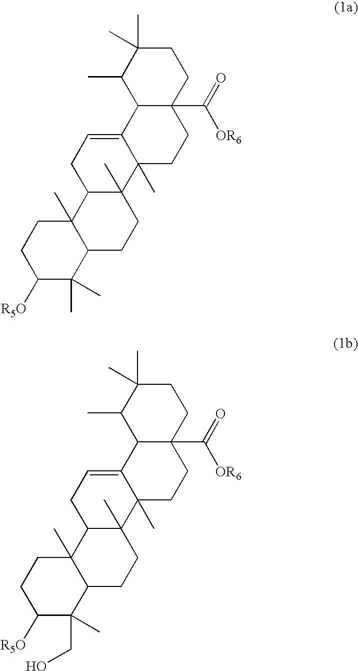

The oleanolic acid represented by the formula (1a) is a saponin compound having oleanolic acid as sapogenin and is known to have anticancer as well as anti-inflammatory effects [Li J, Guo W J, Yang Q Y. Effects of ursolic acid and oleanolic acid on human colon carcinoma cell line HCT15. World J Gastroenterol. 2002 June; 8(3): 493-5], cerebral nerve protecting effect [Qian Y H, Liu Y, Hu H T, Ren H M, Chen X L, Xu J H. The effects of the total saponin of Dipsacus asperoides on the damage of cultured neurons induced by β-amyloid protein 25-35], antiviral effect [Kapil A, Sharma S. Effect of oleanolic acid on complement in adjuvant- and carrageenan-induced inflammation in rats. J. Pharm Pharmacol. 1995 July; 47(7): 585-7], antihyperlipidemic effect [Lee K T, Sohn I C, Kim D H, Choi J W, Kwon S H, Park H J. Hypoglycemic and hypolipidemic effects of tectorigenin and kaikasaponin III in the streptozotocin-Induced diabetic rat and their antioxidant activity in vitro. Arch Pharm Res. 2000 October; 23(5): 461-6], antiallergenic effect [Park K H, Park J, Koh D, Lim Y. Effect of saikosaponin-A, a triterpenoid glycoside, isolated from Bupleurum falcatum on experimental allergic asthma. Phytother Res. 2002 June; 16(4): 359-63], immune control effect [Ju D W, Zheng Q Y, Cao X, Fang J, Wang H B. Esculentoside A inhibits tumor necrosis factor, interleukin-1, and interleukin-6 production induced by lipopolysaccharide in mice. Pharmacology. 1998 April; 56(4): 187-95] and anti-angiogenic effect [Korean Patent No. 101,480].

And, the hederagenin saponin represented by the formula (1b) is a compound having hederagenin as sapogenin and is known to have anti-inflammatory effect [Kwak W J, Han C K, Chang H W, Kim H P, Kang S S, Son K H. Loniceroside C, an antiinflammatory saponin from Lonicera japonica. Chem Pharm Bull (Tokyo). 2003 March; 51(3): 333-5], pain-alleviating effect [Choi J, Huh K, Kim S H, Lee K T, Park H J, Han Y N. Antinociceptive and anti-rheumatoidal effects of Kalopanax pictus extract and its saponin components in experimental animals. J Ethnopharmacol. 2002 February; 79(2): 199-204], antioxidation effect [Choi J, Huh K, Kim S H, Lee K T, Lee H K, Park H J. Kalopanaxsaponin A from Kalopanax pictus, a potent antioxidant in the rheumatoidal rat treated with Freund's complete adjuvant reagent. J Ethnopharmacol. 2002 January; 79(1): 113-8] and blood sugar lowering effect [Kim D H, Yu K W, Bae E A, Park H J, Choi J W. Metabolism of Kalopanaxsaponin B and H by human intestinal bacteria and antidiabetic activity of their metabolites. Biol Pharm Bull. 1998 April; 21(4): 360-5].

But, until now, nothing is known about the effect of the oleanane-type triterpene saponin compound represented by the formula (1), which comprises oleanolic acid saponin or hederagenin saponin, in treating dementia and mild cognitive impairment and improvement of brain functions.

Japanese Patent Laid-Open No. 2000-247993 recognizes the high affinity of oleanolic acid to sigma receptors and discloses that it is effective in treating various brain diseases related with the sigma receptors, including schizophrenia, depression, anxiety, cerebrovascular diseases, behavior disorder in the aged people, Alzheimer's disease, Parkinson's disease, Huntington's disease, drug addiction, stress, etc. However, this patent presents no in vivo animal test or in vitro nerve cell test results directly related with the treatment of the diseases.

The oleanane-type triterpene saponin compounds having oleanolic acid or hederagenin as sapogenin may be in the form of a monodesmoside bound to sugar at C-3 or C-28 position or in the form of a bisdesmoside bound to sugars at both C-3 and C-28 positions. There exist more than 150 saponin compounds from the various combinations of the sugar chain [Kang Sam Sik, Triterpenoid saponin, Seoul National University Press, 1996, TradiMed Database].

Oleanolic Acid Saponins:

Achyranthoside C, acutosides A-G, akeboside Stj, anemoside A, araliasaponins XII-XVIII, araloside D, arvensosides A-B, betavulgarosides IV-V, Bupleurum chinense triterpene glycoside S1, caraganoside A, chikusetsusaponins IB, IV, ciwujianosides A1, C4, C3, D1, clematichinenosides A, C, Clematis chinensis prosapogenins CP9, CP9a, CP7a, CP2b, clemontanosides E, F, saffron (Crocus sativus) oleanolic acid saponin, elatosides B, D, eleutheroside K, fatsiaside A1, hederacolchiside E, hederasaponin B, huzhangosides A, B, C, lablaboside A, lucyoside H, 6′-methylmomordin I, momordins I, IC, IIB, 3-O-α-L-arabinopyranosyloleanolic acid, 3-O-α-L-arabinopyranosyloleanolic acid-28-O-β-D-glucosyl(1→6) β-D-glucoside, 3-O-α-L-rhamnopyranosyl(1→2)-α-L-arabinopyranosyloleanolic acid 28-O-β-D-xylopyranosyl(1→6)-β-D-glucopyranosyl ester, 3-O-β-D-galactopyranosyl(1→2)-β-D-glucolopyranosyloleanolic acid, 3-O-β-D-glucopyranosyl(1→3)-α-L-rhamnopyranosyl(1→2)-α-L-arabinopyranosyloleanolic acid, 3-O-β-D-glucopyranosyl(1→3)-α-L-rhamnopyranosyl(1→2)-α-L-arabinopyranosyloleanolic acid-28-gentiobiocide, 28-O-β-D-glucopyranosyloleanolic acid, 3-O-β-D-glucopyranosyloleanolic acid, 3-O-β-D-glucolopyranosyloleanolic acid, 3-O-β-D-ribopyrosyl(1→3)-α-L-rhamnopyranosyl(1→2)-α-L-arabinopyranosyloleanolic acid, 3-O-β-D-xylopyranosyl(1→3)-α-L-rhamnopyranosyl(1→2)-α-L-arabinopyranosyloleanolic acid, 3-O-β-D-xylosyl(1→4)-β-D-glucosyl(1→4)-α-L-rhamnosyl(1→3)-β-D-glucosyl(1→3)-α-L-rhamnosyl(1→2)-α-arabinosyloleanolic acid 28-O-β-D-glucosyl(1→6)-β-D-glucosyl ester, 2′-O-glucopyranosylmomordin Ic, 2′-O-glucopyranosylmomordin Ic, 3-O-β-D-xylopyranosyl(1→4)-β-D-glucopyranosyl (1→3)-α-L-rhamnopyranosyl(1→2)-α-L-arabinopyranosideoleanolic acid 28-O-β-D-glucopyranosyl(1→6)-β-D-glucopyranosyl ester, oleanolic acid 3-O-neohesperidoside, oleanolic acid 3-O-α-L-arabinopyranosyl-28-O-β-D-glucopyranosyl(1→6)-β-D-glucopyranosyl ester, oleanolic acid-3-O-β-D-glucopyranosyl(1→2)-α-L-arabinopyranoside, pericarpsaponin J3, prosapogenin CP4, quinatoside D, raddeanins A, C, D, E, F, raddeanosides R10, R11, rivularinin, spinacosides C, D, spinasaponin A, tarasaponins II, III, VI, Tetrapanax papyriferum saponins R-3, R-1-a, R-4-b, udosaponin methyl esters A-C.

Hederagenin Saponin:

Akebia saponins A-G, akeboside, asperosaponins F, H1, calcoside D, caulosides D, F, Clematis chinensis prosapogenins CP10, CP10a, Cp8a, CP3b, CP0, CP3a, CP2a, 3-O-β-D-xylopyranosyl(1→3)-α-L-arabinopyranosylhederagenin-28-O-α-L-rhamnopyranosyl(1→4)-β-D-glucopyranosyl(1→6)-β-D-glucopyranoside, hederagenin-3-O-α-L-arabinoside, hederagenin-3-O-[α-L-rhamnopyranosyl-(1→2)-α-L-arabinopyranosyl]-28-O-β-D-xylopyranosyl(1→6)-β-D-glucopyranosyl ester, hederagenin-3-O-[α-L-rhamnopyranosyl(1→2)-α-L-arabinopyranosyl]-28-O-[3-O-acetyl-β-D-glucopyranosyl(1→6)-β-D-glucopyranoside, hederagenin-3-O-[α-L-rhamnopyranosyl(1→2)-α-L-arabinopyranosyl]-28-[3-O-acetyl-β-D-xylopyranosyl(1→6)-β-D-glucopyranoside, hederagenin-3-O-α-L-rhamnopyranosyl(1→3)-β-D-glucopyranosyl(1→3)-α-L-rhamnopyranosyl(1→2)-α-L-arabinopyranosyl 28-O-β-D-glucopyranosyl(1→6)-β-D-glucopyranosyl ester, hederagenin-3-O-β-D-glucopyranosyl(1→3)-α-L-rhamnopyranosyl(1→2)-α-L-arabinopyranosyl 28-O-β-D-glucopyranosyl(1→6)-β-D-glucopyranosyl ester, hederagenin-3-O-(4-O-acetyl)-α-L-arabinopyranosyl 28-O-β-D-glucopyranosyl(1→6)-β-D-glucopyranosyl ester, kalopanaxsaponins B, G, JLa, JLb, leontosides A, B, lucyosides A, E, mukurozisaponins EI, G, X, Y2, YI, 4′-O-acetylakebiasaponin D, 3-O-[α-L-arabinosyl]hederagenin-28-O-[β-D-glucosyl]ester, 3-O-α-L-rhamnopyranosyl (1→3) β-D-glucosyl(1→3)-α-D-rhamnosyl (1→2)-α-arabinosylhederagenin-28-O-β-D-glucosyl(1→6) β-D-glucosyl ester, 3-O-β-D-glucopyranosyl (1→3)-α-L-rhamnopyranosyl (1→2)-α-L-arabinopyranosylhederagenin, 3-O-β-D-glucopyranosylhederagenin, 3-O-(β-D-glucosyl(1→4)-α-L-rhamnosyl(1→3) β-D-glucosyl(1→3)-α-D-rhamnosyl(1→2)-α-arabinosylhederagenin-28-O-β-D-glucosyl(1→6)-β-D-glucosyl ester, 3-O-(β-D-glucosyl(1→4)-α-L-rhamnosyl(1→3)-β-D-glucosyl(1→3)-α-L-rhamnosyl(1→2)-α-arabinosylhederagenin, 3-O-(β-D-xylosyl(1→4) β-D-glucosyl(1→4)-α-L-rhamnosyl(1→3)-β-D-glucosyl(1→3)-α-L-arabinosyl(1→2)-α-arabinosylhederagenin, 3-O-[(2′-O-acetyl)-α-L-arabinopyranosyl(1→6)-β-D-glucopyranosyllhederagenin, 3-O-(2′-O-acetyl)-α-L-arabinopyranosylhederagenin-28-O-[β-D-glucopyranosyl(1→6)-β-D-glucopyranoside], percarpsaponins C, J2, G, K, pulsatilosides A, B, C, quinatosides A, B, C, sapindosides A, B, C, staunosides A, B, D, E, tauroside G3, udosaponin methyl esters D, E, F.

The oleanane-type triterpene saponin compounds comprising oleanolic acid saponin and hederagenin saponin are extracted from the following plants: plants in the genus Amaranthus, e.g., Achyranthes sp., Amaranthus sp., etc.; plants in the Araliaceae family, e.g., Acanthopanax sp., Aralia sp., Fatsia sp., Kalopanax sp., Panax sp., Tetrapanax sp., etc.; plants in the Basellaceae family, e.g., Boussingaultia sp., etc.; plants in the Berberidaceae family, e.g., Caulophyllum sp., etc.; plants in the Boraginaceae family, e.g., Anchusa sp., etc.; plants in the Caprifoliaceae family, e.g., Lonicera sp., etc.; plants in the Chenopodiaceae family, e.g., Chenopodium sp., etc.; plants in the Cucurbitaceae family, e.g., Actinostemma sp., Luffa sp., Momordica sp., etc.; plants in the Dipsacaceae family, e.g.; Dipsacus sp., etc.; plants in the Euphorbiaceae family, e.g., Putranjiva sp., etc.; plants in the Hippocastanaceae family, e.g., Aesculus sp., etc.; plants in the Lardizabalaceae family, e.g., Akebia sp., etc.; plants in the Leguminosae family, e.g., Acacia sp., Albizzia sp., Swartzia sp., etc.; plants in the Opiliaceae family, e.g., Opilia sp., etc.; plants in the Phytolaccaceae family, e.g., Phytolacca sp., etc.; plants in the Ranunculaceae family, e.g., Anemone sp., Clematis sp., Hedera sp., Pulsatilla sp., etc.; plants in the Rubiaceae family, e.g., Randia sp., Xeromphis sp., etc.; plants in the Sapindaceae family, e.g., Pometia sp., Spindus sp., Thinouia sp., etc.; plants in the Valerianaceae family, e.g., Patrinia sp.

The present inventors performed animal tests and the result reveals that, as compared to the control group where no drug was administered, the group where scopolamine (1 mg/kg), which is known to decline the memory function by inhibiting the transfer of neurotransmitters, was administered and the group where the oleanane-type triterpene saponin compound represented by the formula (1) was administered 1 hour after the administration of scopolamine, the effect of a significant memory improvement was observed.

Accordingly, the oleanane-type triterpene saponin compound represented by the formula (1) can be used as an effective ingredient of a pharmaceutical drug for treating dementia and mild cognitive impairment or as a health food for improving cognitive function.

The drug comprising the oleanane-type triterpene saponin compound represented by the formula (1) as an effective ingredient may be presented in the form of general drug capable of oral or non-oral administration. In the preparation of the drug for oral or non-oral administration, a commonly used pharmaceutically or sitologically acceptable diluent or an excipient such as a filler, an expander, a binder, a wetting agent, a disintegrator, a surfactant, and the like may be used.

Examples of solid drugs for oral administration are a tablet, a pill, powder, a granule, a capsule, and the like. The solid drugs are prepared by mixing at least one excipient, such as starch, calcium carbonate, sucrose, lactose, gelatin, etc., with lignan, lactone or a derivative thereof. Further, such surfactant as magnesium stearate and talc may be added in addition to the excipient. Examples of liquid drugs for oral administration are suspension, liquid medicine for internal use, emulsion, syrup, etc. In addition to simple diluent such as water and liquid paraffin, various excipients, for example, wetter, sweetener, fragrance, preservative, etc., may be included.

Examples of drugs for non-oral administration are sterilized aqueous solution, water-insoluble solution, suspension, emulsion, lyophilized drug and suppository. The water-insoluble solution or suspension includes propylene glycol, polyethylene glycol, vegetable oil such as olive oil, injectable ester such as ethyl oleate, etc. Witepsol, macrogol, Tween 61, cacao butter, laurin fat, glycerol gelatin, etc. may be used as the base of the suppository.

The content of the effective ingredient in the drug may be adequately selected considering the degree of absorption in the body, degree of inactivation, rate of excretion, age, sex, physical conditions of the user, etc. A recommended dosage is 0.1-10 mg/kg/day, based on the oleanane-type triterpene saponin compound represented by the formula (1), more preferably 0.5-5 mg/kg/day. The drug may be administered 1 to 3 times a day.

Also, the oleanane-type triterpene saponin compound represented by the formula (1) may be provided in the form of health food comprising the compound as an active ingredient. In this description, the term “health food” refers to a general food or one prepared in the form of a capsule, powder, a suspension, etc. in which the oleanane-type triterpene saponin compound represented by the formula (1) has been added. When it is intaken, the health food provides a particular health-related effect. But, unlike normal medicines, it does not cause any side effects even after a long-term use because it is prepared by means of food.

### DESCRIPTION OF DRAWINGS

FIG. 1 is a graph showing the effect of the oleanane-type triterpene saponin compounds of the present invention in improvement of memory ability after they were administered once orally.

### BEST MODE

Practical and preferred embodiments of the present invention will be illustrated as shown in the following examples. However, it will be appreciated that those skilled in the art may, in consideration of this disclosure, make modifications and improvements within the spirit and scope of the present invention.

### Example 1

### Extraction of Oleanane-Type Triterpene Saponin

Oleanolic acid saponin and hederagenin saponin were isolated as follows from the following herbs known to contain lots of oleanolic acid saponin and hederagenin saponin.

Each 1 kg (dry weight) of Aralia elata, Pulsatilla chinensis, Lonicera japonica and Kalopanax pictus was extracted with 7 L of 50% ethanol for 4 hours while refluxing. This procedure was repeated for 2 times. The extract was filtered and concentrated under reduced pressure at 50° C. using a rotary evaporator. Water was added to the resultant condensate to a volume of about 5 equivalents (V/W). The resultant suspension was mixed with water saturated n-butanol of equal volume, put in a separation funnel, stirred and let alone for 24 hours. The above butanol layer was isolated. After 2-3 times of re-fractionation, the butanol fraction was concentrated using an evaporator and the solvent was completely removed in a vacuum oven.

Column chromatography was performed on the n-butanol fraction using octadecylsilylated silica resin (YMC*GEL ODS-A 12 nm, S-150 m). The amount of the resin was 250 g, or 25 equivalents of the sample amount (10 g). The step-gradient method of stepwise increasing the methanol content of solvent from 10% (V/V) methanol, which amounts to 2-3 volume equivalents of the resin, by 10% (V/V) was employed. Only the fractions isolated by 70%, 80% and 90% methanol solvents (V/V) were taken to concentrate the saponin compound as much as possible.

### Example 2

### Isolation and Structure Analysis of Oleanane-Type Triterpene Saponin

High performance liquid chromatography (HPLC) was performed on the methanol fractions using acetonitrile/water mixture solvent. Using a PDA detector, major peaks having maximum absorptivity at 210 nm and showing the specific triterpene absorption spectra were selected. Subsequently, preparative HPLC was performed using acetonitrile/water mixture solvent of 9.5 mL/min to separate the selected peaks. After concentration using an evaporator, the solvent was completely removed by drying in a vacuum oven. YMC J'Sphere ODS-H80 column was used and component analysis was performed at 210 nm. The isolated peak components were hydrolyzed in acidic condition by heating at 100° C. in 2N H₂SO₄ for 60 minutes to remove sugar from saponin. The resultant product was analyzed by HPLC along with oleanolic acid and hederagenin standard substances purchased from Sigma. The peak components confirmed as oleanolic acid saponin or hederagenin saponin were further separated to obtain the 8 substances listed in Table 1 below.

¹H-NMR (500 MHz), ¹³C-NMR (125 MHz), DEPT and 2D NMR (1H-1H COSY, HMQC, HMBC, TOCSY, NOESY) analyses were performed to determine the structure of the isolated substances. And, the hydrolyzed sugars were analyzed with the carbohydrate analysis system and analyzed by GC after TMS-derivatization, in order to identify the identity and number of the sugars. The structure of each sugar was performed by 2D NMR (¹H-¹H COSY, HMQC, HMBC, TOCSY) analysis. The binding of the sugars was analyzed by 2D NMR(HMBC, NOESY).

100-500 mg of 8 oleanolic acid saponin or hederagenin saponin substances were obtained as given in Table 1.

TABLE 1

| | | | Molecular | |
|---|---|---|---|---|
| Category | Scientific Names | Herbs | formula | M.W. |
| Substance \textcircled{1} | Eleutheroside K | Pulsatilla | C₄₁H₆₆O₁₁ | 735.0 |
| | 3-O-α-L-Rhamnopyranosyl(1→2)-α-L-arabinopyranosyloleanolic | chinensis | | |
| | acid | | | |
| Substance \textcircled{2} | Hederasaponin B | Pulsatilla | C₅₉H₉₆O₂₅ | 1205.4 |
| | 3-O-α-L-Rhamnopyranosyl(1→2)-α-L-arabinopyranosyloleanolic | chinensis | | |
| | acid-28-O-α-L-rhamnopyranosyl(1→4)-β-D-glucopyranosyl(1→6)- | | | |
| | β-D-glucopyranosyl ester | | | |
| Substance \textcircled{3} | Hederacolchiside E | Pulsatilla | C₆₅H₁₀₆O₃₀ | 1366.6 |
| | 3-O-α-L-Rhamnopyranosyl(1→2)-β-D-glucopyranosyl(1→4)-α-L- | chinensis | | |
| | arabinopyranosyloleanolic acid-28-O-α-L-rhamnopyranosyl(1→4)- | | | |
| | β-D-glucopyranosyl(1→6)-β-D-glucopyranosyl ester | | | |
| Substance \textcircled{4} | Elatoside A | Aralia | C₄₇H₇₄O₁₈ | 927.1 |
| | 3-O-β-D-Xylopyranosyl(1→2)-β-D-galactopyranosyl(1→3)-β-D- | elata | | |
| | glucolopyranosyloleanolic acid | | | |
| Substance \textcircled{5} | Elatoside C | Aralia | C₅₃H₈₄O₂₃ | 1089.2 |
| | 3-O-β-D-Xylopyranosyl(1→2)-β-D-galactopyranosyl(1→3)-β-D- | elata | | |
| | glucolopyranosyloleanolic acid-28-O-β-D-glucopyranosyl ester | | | |
| Substance \textcircled{6} | Loniceroside A | Lonicera | C₅₂H₈₄O₂₁ | 1045.2 |
| | 3-O-α-L-Arabinopyranosylhederagenin-28-O-α-L- | japonica | | |
| | rhamnopyranosyl(1→2)-β-D-xylopyranosyl(1→6)-β-D- | | | |
| | glucopyranosyl ester | | | |
| Substance \textcircled{7} | Loniceroside B | Lonicera | C₅₈H₉₄O₂₅ | 1191.4 |
| | 3-O-α-L-Rhamnopyranosyl(1→2)-α-L- | japonica | | |
| | arabinopyranosylhederagenin-28-O-α-L-rhamnopyranosyl(1→2)-β- | | | |
| | D-xylopyranosyl(1→6)-β-D-glucopyranosyl ester | | | |
| Substance \textcircled{8} | Kalopanaxsaponin B, Pericarpsaponin Pk | Kalopanax | C₅₉H₉₆O₂₆ | 1221.4 |
| | 3-O-α-L-Rhamnopyranosyl(1→2)-α-L- | pictus | | |
| | arabinopyranosylhederagenin-28-O-α-L-rhamnopyranosyl(1→4)-β- | | | |
| | D-glucopyranosyl(1→6)-β-D-glucopyranosyl ester | | | |

### Example 3

### Passive Avoidance Test

Passive avoidance test was performed to identify the memory improvement effect of the above 8 oleanolic acid saponin or hederagenin saponin substances in the body. Also, oleanolic acid and hederagenin, aglycons of the saponin compounds, were included in the test.

A shuttle box measuring 50 cm×15 cm×40 cm was used. The box was divided into two compartments by a guillotine door. One compartment was brightly illuminated and the other was covered by black cloth.

A mouse was placed in the illuminated compartment. Then, when the guillotine door was open, the mouse moved into the dark compartment within 20 seconds because it prefers darkness. The guillotine door was closed automatically as soon as the mouse entered the dark compartment. In this way, the latency time, or the time that elapsed until a mouse left the illuminated compartment and entered the dark compartment, was determined. On the first day, this training trial was performed until all the mice entered the dark compartment within 20 seconds.

On the following day, the trained mice were placed in the illuminated compartment, one at a time, and were allowed to move into the dark compartment. When the mice entered the dark compartment, a foot-shock (0.8 mA, 3 seconds) was delivered through the electronic grid equipped on the floor of the dark compartment.

24 hours after this acquisition trial, the mice were placed in the illuminated compartment and were allowed to move into the dark compartment. Then, normal mice hesitated to move into the dark compartment remembering the shock of the day before. The time elapsed until the mice entered the dark compartment was measured up to 300 seconds.

Retention time was measured to evaluate the memory improvement effect. Neither scopolamine nor the drug was administered to the control group. Scopolamine (1 mg/kg), which is known to decline memory function by inhibiting the transfer of neurotransmitters, was administered. An hour later, saponin (30 mg/kg) and aglycon (30 mg/kg), Aricept (donepezil, 1 mg/kg, positive control) and water (negative control) were administered.

The negative control group to which Aricept had been administered orally showed 1.6 times longer retention time than the negative control group to which water had been administered. Although there were some variations depending on the compositions of aglycon and sugar, all the 8 oleanolic acid saponin or hederagenin saponin substances in accordance with the present invention showed about 2.5-3.0 times longer retention time than that of the negative control group.

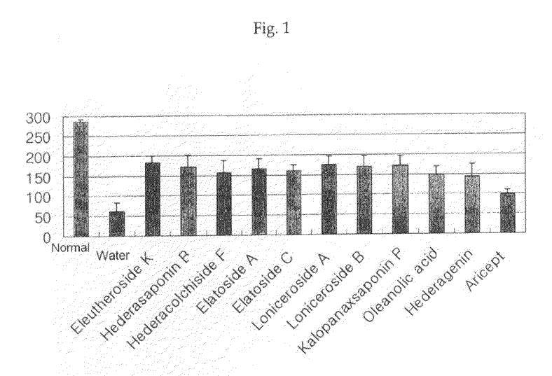

Further, these saponins showed a better cognitive function improvement effect than the aglycons oleanolic acid (2.4 times) and hederagenin (2.3 times) [see Table 2 and FIG. 1].

Thus, it can be concluded that oleanolic acid saponin and hederagenin saponin are effective for improving memory which is declined in dementia and mild cognitive impairment. They are superior in the effect not only to Aricept, which is used to treat dementia, but also to oleanolic acid and hederagenin, which are aglycons of saponin.

TABLE 2

| Treatment | | Retention time (sec) |
|---|---|---|
| Normal | | 286.9 ± 5.8 |
| Water | | 61.5 ± 22.4 |
| Oleanolic acid | | 149.4 ± 19.7 |
| Hederagenin | | 142.8 ± 32.6 |
| Aricept | | 98.5 ± 10.6 |
| Oleanolic acid saponins | Eleutheroside K | 182.6 ± 15.9 |
| | Hederasaponin B | 172.3 ± 29.1 |
| | Hederacolchiside E | 155.7 ± 31.2 |
| | Elatoside A | 166.0 ± 24.8 |
| | Elatoside C | 160.1 ± 15.6 |
| Hederagenin saponins | Loniceroside A | 175.3 ± 21.1 |
| | Loniceroside B | 169.7 ± 25.4 |
| | Kalopanaxsaponin B | 170.5 ± 24.6 |

Presented below are the examples of preparing therapeutic pharmaceutical drugs or health foods that comprise the oleanane-type triterpene saponin compound represented by the formula (1) as an active ingredient. However, they should not be construed as limiting the scope of the present invention.

### Preparation Example 1

### Preparation of Powder and Capsule

50 mg of the oleanane-type triterpene saponin compound was mixed with 74 mg of lactose, 15 mg of crystalline cellulose and 1 mg of magnesium stearate to obtain power. The resultant powder was filled into a No. 5 gelatin capsule using an adequate apparatus.

### Preparation Example 2

### Preparation of Liquid Drug

50 mg of oleanane-type triterpene saponin compound was added to 20 g of sugar, 20 g of isomerized sugar and adequate amount of lemon flavor. Sterilized purified water was added to a total volume of 100 mL. The resultant liquid was filled into a brown bottle and sterilized.

### Preparation Example 3

### Preparation of Health Food

100 mg of the oleanane-type triterpene saponin compound represented by the formula (1) were mixed with 100 mg of ginseng extract, 100 mg of green tea extract, 100 mg of vitamin C, 120 mg of powdered vitamin E, 2 mg of ferrous lactate, 2 mg of zinc oxide, 20 mg of nicotinamide, 5 mg of vitamin A, 2 mg of vitamin B1, 2 mg of vitamin B2, 200 mg of cornstarch and 20 mg of magnesium stearate (for one day).

### Industrial Applicability

As apparent from the above description, the oleanane-type triterpene saponin compound represented by the formula (1) has superior memory improvement effect and, thus, is useful for drugs for treating dementia and mild cognitive impairment and health foods for the improvement of brain functions, including cognitive function.

Those skilled in the art will appreciate that the concepts and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the present invention as set forth in the appended claims.

1. A drug effective for the treatment and prevention of dementia and mild cognitive impairment which comprises an oleanane-type triterpene saponin compound represented by the formula (1) as an effective ingredient: 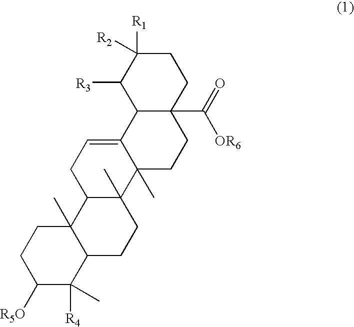
wherein each of R₁, R₂ and R₃ is hydrogen or C₁-C₄ alkyl; R₄ is C₁-C₄ alkyl or C₁-C₄ hydroxyalkyl; and each of R₅ and R₆ is hydrogen or sugar, wherein at least one of R₅ and R₆ is sugar which is selected from glucose, galactose, rhamnose, xylose, arabinose and glucuronic acid.

2. The drug as set forth in claim 1, wherein the compound represented by the formula (1) is an oleanane-type triterpene saponin compound selected from the group consisting of eleutheroside K, hederasaponin B, hederacolchiside E, elatoside A, elatoside C, loniceroside A, loniceroside B and kalopanaxsaponin B.

3. A health food effective for the improvement of brain functions which comprises an oleanane-type triterpene saponin compound represented by the formula (1): 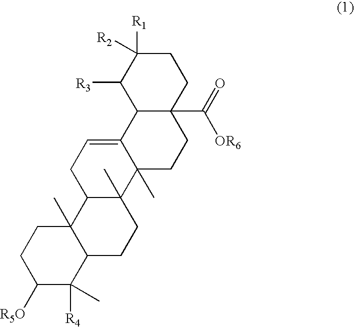
wherein each of R₁, R₂ and R₃ is hydrogen or C₁-C₄ alkyl;. R₄ is C₁-C₄ alkyl or C₁-C₄ hydroxyalkyl; and each of R₅ and R₆ is hydrogen or sugar, wherein at least one of R₅ and R₆ is sugar which is selected from glucose, galactose, rhamnose, xylose, arabinose and glucuronic acid.

4. The health food as set forth in claim 1, wherein the compound represented by the formula (1) is an oleanane-type triterpene saponin compound selected from the group consisting of eleutheroside K, hederasaponin B, hederacolchiside E, elatoside A, elatoside C, loniceroside A, loniceroside B and kalopanaxsaponin B.
